(19) United States
(12) Patent Application Publication
(10) Pub. No.: US 2021/0322382 A1
(43) Pub. Date: Oct. 21, 2021

(54) METHODS AND COMPOSITIONS FOR IMPROVED TREATMENT OF SINUS DISEASE

(71) Applicant: Veloce BioPharma LLC, Fort Lauderdale, FL (US)

(72) Inventors: Joseph CAPRIOTTI DECIO, Arrecife (ES); Belachew TESSEMA, Framington, CT (US)

(73) Assignee: Veloce BioPharma LLC, Fort Lauderdale, FL (US)

(21) Appl. No.: 16/892,218

(22) Filed: Jun. 3, 2020

Related U.S. Application Data
(60) Provisional application No. 63/021,035, filed on May 6, 2020.
(60) Provisional application No. 63/021,039, filed on May 6, 2020.
(60) Provisional application No. 63/015,313, filed on Apr. 24, 2020.
(60) Provisional application No. 63/012,629, filed on Apr. 20, 2020.
(60) Provisional application No. 63/011,961, filed on Apr. 17, 2020.

(30) Foreign Application Priority Data
May 20, 2020 (NL) 2025640
May 20, 2020 (NL) 2025641

(51) Int. Cl. A61K 31/4015; A61K 33/18; A61K 47/38; A61K 9/00

(52) U.S. Cl. CPC A61K 31/4015; A61K 9/0043; A61K 47/38; A61K 33/18

(57) ABSTRACT

Compositions for treating, controlling, reducing, or inhibiting a chronic established biofilm in a sinus of a human when applied topically or through irrigation is disclosed. Methods for treating a subject with a bacterial infection or a SARS-CoV-2 infection using a disclosed composition as disclosed, the composition comprising 0.5 wt %-2.5 wt % povidone-iodine; 0.15 wt %-1.25 wt % hydroxyethylcellulose; and water.

### PRIORITY

This application claims the benefit of priority to Netherlands Patent Application No. 2025640, filed May 20, 2020 and entitled “Methods and composition for improved antisepsis;” and Netherlands Patent Application No. 2025641, filed May 20, 2020 and entitled “Methods and compositions for improved treatment of sinus disease;” U.S. Provisional Patent Application Ser. No. 63/021,035, filed May 6, 2020 and entitled “Methods and compositions for the treatment of Covid-19;” U.S. Provisional Patent Application Ser. No. 63/021,039, filed May 6, 2020 and entitled “Methods and compositions for improved treatment of sinus disease;” U.S. Provisional Patent Application Ser. No. 63/015,313, filed Apr. 24, 2020 and entitled “Compositions and methods for improved treatment of sinus disease;” U.S. Provisional Patent Application Ser. No. 63/012,629, filed Apr. 20, 2020 and entitled “Compositions and methods for improved antiseptics;” and U.S. Provisional Patent Application Ser. No. 63/011,961, filed Apr. 17, 2020 and entitled “Compositions and methods for the treatment of Covid-19.” The entire contents of each of the above listed priority patent applications are incorporated herein by reference.

### BACKGROUND

Topical antibiotics are an important part for the management of CF-CRS (cystic fibrosis-rhinosinusitis) given the low rate of systemic effects and ability to deliver higher concentrations directly to the infected sinuses. Several studies have established topical antibiotics to be efficacious in patients with cystic fibrosis population. Intranasal tobramycin has perhaps been the most well-studied antibiotic and has been associated with significant improvements in quality of life, reduced Paeruginosa colonization of the sinuses, and improvement in sinus inflammation on serial MRI. It is important that many of these studies investigated the use of topical antibiotics in patients who had undergone prior endoscopic sinus surgery (ESS) given the significant improvement in the delivery of topical medications to the sinuses after surgery. Topical antibiotics have also shown efficacy in the immediate postoperative period.

The literature has shown benefits of antibiotics postoperatively. Aanaes and colleagues investigated 61 treated post-ESS patients with 2 weeks of IV antibiotics directed at colonizing bacteria in addition to 6 months of colistin nasal irrigations and 12 months of topical nasal steroids. Following this regimen, the investigators demonstrated eradication of pathogenic bacteria in 67% of study patients at 6 months postoperative. Topical antibiotic use is another adjunctive therapy for patients with CF after ESS. As stated earlier, studies using topical antibiotic and steroid rinses as part of a post-ESS regimen have shown high rates of eradication of pathogenic bacteria in the sinuses and lungs, significant improvements in symptoms and endoscopy scores, reduced number of hospitalizations from pulmonary exacerbations, and even a reduction in revision or salvage surgeries.

Antiseptics are crucial for the practice of medicine; however, currently used antiseptics still have room for improvement and still have drawbacks such as significant toxicity, failure rate and chemical instability. These drawbacks limit their effectiveness and wider use. Many antiseptics are toxic to human respiratory cells, ciliated epithelial cells and Goblet cells. Antiseptics are commonly used prior to routine phlebotomy, in preparation for minor and major invasive procedures, and as part of routine infection control hand-washing practices. Intranasal antiseptic use is limited and only described for single-use as presurgical decontamination. The failure and toxicity of antiseptics, and nasal antiseptics in particular, often result in nosocomial infections, cellular damage, dysfuntion of respiratory epithelium, ineffective reduction of viral shedding in the nasal passages and increased transmission of viral diseases. Antiseptic failure can also contribute to the transmission of viral disease, including the SARS-CoV-2 (sinusitis, rhinosinusitis, chronic sinus disease associated with cystic fibrosis) virus and can worsen outbreaks. Failed or incomplete antisepsis of human tissues such as the nasal passages can lead to the spread of respiratory disease and sinusitis, rhinosinusitis, and chronic sinus disease associated with cystic fibrosis. The use of existing antiseptics in the nasal passages can be toxic to the nasal mucosa. Failed or incomplete antisepsis of surfaces such as human skin, medical devices, tabletops, everyday objects, door handles, and any other objects touched by humans can lead to the spread and worsening outbreaks of viral diseases, including COVID-19 and respiratory diseases associated with cystic fibrosis.

Many antibiotics and antiseptics, when used alone for CF-CRS, are not as effective as desired. Their disadvantages include requiring long contact times, being toxic and irritating to patients, or their ineffectiveness against biofilms. “Biofilm” refers to an extracellular matrix in which microorganisms are dispersed and/or form colonies. The biofilm typically is made of polysaccharides and other macromolecules. Further, many antiseptics and antibiotics require prolonged contact times with sinus tissues to be effective. Such contact times are often difficult to achieve and are toxic to the nasal mucosa. Other reasons for the ineffectiveness of antibiotics and antiseptics include: (1) inability to penetrate into the sinus mucosa; (2) requirement for steroids or anti-inflammatories to reduce inflammation; (3) instability in a pharmaceutical composition. Also, many antiseptics are toxic to the nasal mucosal cells and cannot be used at concentrations high enough to provide adequate antisepsis. Thus, there remains a significant need for improved antiseptics.

Therefore, there is a need for an improved composition without steroids could lower rates of symptomatic sinusitis, rhinosinusitis, chronic sinus disease associated with cystic fibrosis. Improved antiseptics and antibiotics compositions would also enable the improved treatment of sinusitis, rhinosinusitis, chronic sinus disease associated with cystic fibrosis. Improved nasal antiseptics could reduce aerosolization from the sinonasal passages and could reduce viral transmission from aerosolized droplets in the sinonasal passages.

### SUMMARY

The invention relates to stable topical compositions and methods using such compositions useful in the treatment of human tissues; treatment of human tissue surfaces such as skin, epithelium and mucosal surfaces; treatment of chronic sinusitis, treatment of acute sinusitis, treatment of rhinosinusitis, treatment of infections associated with cystic fibrosis, and treatment of biofilms in human disease.

The invention also relates to stable topical compositions useful in the treatment of viral, bacterial fungal and protozoal infections of the skin, mucosa, epithelium, nasal passages, oropharynx, nasopharynx, upper airway, lower airway and other human surfaces. One aspect of the invention relates to compositions and methods to reduce viral shedding, viral replication and viral transmission of SARS-CoV-2, other coronaviruses and viruses other than coronaviruses. Another aspect of the invention relates to the method of reducing the number of virus particles detectable by PCR, CC, IFA and other viral detection methods from cultured cells, from the respiratory epithelium, from the upper airway, from the lower airway and other human tissues with said compositions. Another aspect of the invention relates to treating viral, bacterial, fungal, protozoal and idiopathic infectious diseases of the respiratory epithelium, upper airway, lower airway and other human tissues with said compositions in patients who also suffer from cystic fibrosis. Another aspect of the invention also relates to treating viral, bacterial, fungal, protozoal and idiopathic infectious diseases of the respiratory epithelium, upper airway, lower airway and other human tissues with said compositions in patients who do not have cystic fibrosis.

The disclosed composition, which is one aspect of the invention, can be, or be incorporated into, a drug, antiseptic, nasal spray, intranasal gel, inhaled agent, rinse, gargle, flush or other pharmaceutical forms. The composition can be used alone or it can be used in addition to other agents for the relief of signs and symptoms of sinusitis, rhinosinusitis, chronic sinus disease associated with cystic fibrosis or occurring in the absence of cystic fibrosis. The invention can be a drug, formulation, antiseptic, nasal spray, intranasal gel, rinse, flush, powder or other pharmaceutical forms which can be incorporated into pads, wipes, cloths, sachets, swabs, woven materials, synthetic materials, sponges, clothing fibers, masks, gloves and other objects where reduction of contamination is desired.

One embodiment relates to a composition comprising DMSO, water, povidone-iodine (PVP-I) and a cellulosic polymer. Optionally, this composition may comprise an alcohol. The alcohol may be, for example, ethanol or isopropyl alcohol. Another embodiment relates to a composition comprising PVP-I, water and cellulosic polymers but without DMSO and without alcohol. In any embodiment, the cellulosic polymer may be hydroxyethylcellulose. In any embodiment, the PVP-I may be at a concentration between 0.5% and 5%. In any embodiment, the DMSO, may be at a concentration of between 0 and 30%. In any embodiment, the cellulosic polymer or polymers may be at a concentration between 0% to 30%, such as 0.25% and 3.0%. The composition of the invention includes any composition disclosed and includes, at least, the compositions in this paragraph.

Another embodiment is directed to a pharmaceutical composition comprising any of the composition of this disclosure.

Another embodiment is directed to a method of treating a patient in need of treatment for a microorganism infection, the method comprising a step of administering a composition of the invention to the surface of a tissue of said patient wherein the tissue has the microorganism infection. The administering may be by irrigation, topical administration, inhalation, nasal mist, nebulizer, atomizer, nasal swab or nasal lavage. In one aspect, the subject may have a disease such as cystic fibrosis. In another aspect, the subject can have one or more diseases selected from the group consisting of: chronic sinusitis, acute sinusitis; rhinosinusitis; infections; sinus infection; and a combination thereof.

For any of the embodiments, the microorganism to be treated by the composition or the method for treating the microorganism infection may be a microorganism selected from the group consisting of a bacteria, a virus, a fungus, a protozoa, an idiopathic infectious disease or a combination thereof. In a preferred embodiment, the microorganism is SARS-CoV-2. Other microorganisms that may be treated by the compositions and methods of the invention are listed in other parts of this disclosure.

In any of the methods, the surface may be a surface selected from the group consisting of skin surface, mucosal tissue surface, nasal tissue surface, oral cavity surface, sinus tissue surface, epithelial tissue surface, a nasal cavity surface, a nasal passage surface, a nasopharynx surface, an oropharynx surface, and a combination thereof. The compositions and methods disclosed can reduce the number of microorganisms on the surface. The compositions and methods disclosed can reduce microorganism shedding, reduce microorganism replication and reduce microorganism transmission, or a combination thereof.

In any of the methods, administering may comprise contacting the composition of the invention to a surface for a period of time between 5 seconds and 120 seconds. In any of the composition and methods, the microorganism infection is an infection of the respiratory epithelium. In any of the composition and methods, the composition has the surprising ability to penetrates through the surface (e.g., the surface of the subject).

Another embodiment is directed to a device (delivery system) comprising the composition. In any of the administration method, administering may be administered through a device (delivery system). The device (delivery system) may be, for example, a nasal swab.

For any embodiment, where a surface is mentioned, it is understood that the surface may be a human surface or a non-human surface.

Another embodiment is directed to a delivery system for delivering a composition of the invention. In other words, the embodiment is directed to a delivery system comprising any composition described in this disclosure. In one aspect, the delivery system may be a compressible liquid plastic blister.

Another embodiment is directed to a method for reducing viral shedding, reducing aerosolization, reducing infection, reducing transmission of sinusitis, reducing rhinosinusitis, or reducing chronic sinus disease in a subject. The method comprises the step of administering any composition embodiment of the invention to a sinonasal surface, a respiratory epithelium surface, or an oral epithelial surface of the subject. The composition may be a pharmaceutical composition—that is—a pharmaceutical composition comprising a composition embodiment of the invention is also an embodiment.

One embodiment is directed to a composition for treating, controlling, reducing, or inhibiting a chronic established biofilm in a sinus of a human when applied topically or through irrigation. While any composition disclosed is effective, preferred embodiments relate to 8 example compositions. The first example composition comprises 0.5 wt % to 1.25 wt % povidone-iodine, 0.5 wt % to 1 wt % hydroxyethylcellulose, and water. The second example composition consists of 0.5 wt % to 1.25 wt % povidone-iodine, 0.5 wt % to 1 wt % hydroxyethylcellulose, and water. The third example composition comprises 0.5 wt % to 1.25 wt % povidone-iodine, 0.5 wt % to 1.25 wt % hydroxyethylcellulose, 0.1 to 2.5% DMSO, and water. The fourth example composition consists of 0.5 wt % to 1.25 wt % povidone-iodine, 0.5 wt % to 1.25 wt % hydroxyethylcellulose, 0.1 to 2.5% DMSO, and water. The fifth example composition comprises 0.5 wt %-2.5 wt % povidone-iodine; 0.15 wt %-1.25 wt % hydroxyethylcellulose; and water. The sixth example composition consists of 0.5 wt %-2.5 wt % povidone-iodine; 0.15 wt %-1.25 wt % hydroxyethylcellulose; and water. The seventh example composition comprises 0.5 wt %-2.5 wt % povidone-iodine; 0.15 wt %-1.25 wt % hydroxyethylcellulose; 0.1 wt % to 2.5 wt % DMSO, and water. The eight example composition consists of 0.5 wt %-2.5 wt % povidone-iodine; 0.15 wt %-1.25 wt % hydroxyethylcellulose; 0.1 wt % to 2.5 wt % DMSO, and water. In one embodiment, any of these compositions, including the compositions containing “consisting of” language, the composition may optionally contain a suitable amount of pharmaceutically acceptable halide-salt to make the solution iso-osmotic with nasal mucosa.

Another embodiment is directed to a method for treating, controlling, reducing, inhibiting or preventing a bacterial infection in a human, the method comprises the step of contacting a site of the bacterial infection with a composition of this disclosure, including, at least, the first example composition, the second example composition, the third example composition, the fourth example composition, the fifth example composition, the sixth example composition, the seventh example composition, or the eight example composition. In the method, the human may have, for example, cystic fibrosis and chronic sinusitis associated with biofilm formation. The step of contacting may involve irrigating the sinus of the human with a composition of claim 1 with one of the example compositions. As a non-limiting illustrative example, the bacterial infection may be a chronic established biofilm in a sinus of a human.

Another embodiment is directed to a method for treating, controlling, reducing, inhibiting or preventing a virus infection in a human, the method comprises the step of contacting a site of the virus infection or potential virus infection with a composition. While any composition disclosed is effective, preferred embodiments relate to one of the 4 example compositions as described. In a preferred embodiment, the virus is SARS-CoV-2.

Each of the features described in this disclosure, for example, for the methods and the compositions, may be combined with any other feature unless otherwise specified.

### DETAILED DESCRIPTION OF THE INVENTION

Topical antibiotics represent a great option to manage the continued biofilm of patients with chronic sinusitis. While studies have shown that PVP-I is effective in the management of patients with CRS, other studies have also shown that PVP-I can be toxic to the nasal mucos, the sinuses and the sinonasal passages. We have been using PVP-I in combination with budesonide twice daily for postoperative cases and for CF for management of the CRS. It has long been known in the art that steroid and anti-inflammatory drugs have an important role in the management of CRS. Steroid-free protocols are known to be less effective than steroid-containing regimes.

There is a clinical need for improved treatments of CRS especially in CF patients. Current antibiotics suffer from antibiotic resistance which develops in many microorganisms. Current protocols with PVP-I require the use of steroids to improve inflammation and are not tolerable and are also less than ideal. We have surprisingly found that by combining small concentrations of PVP-I, optionally with (i.e., with and without) DMSO, and optionally with (i.e., with and without) hydroxyethylcellulose (HEC), we are able to radically improve response in CF-CRS even without steroids. Therefore, one embodiment of this invention is a method of using a composition described in this disclosure as a treatment or CRS-C chronic sinus disease associated with cystic fibrosis.

Povidone-iodine (PVP-I) is a common antiseptic with limited utility due to its undesirable effects such as (1) staining, (2) low viscosity in aqueous solution, (3) toxicity at high concentrations, and (4) difficulty to prepare in a stable form at low concentrations, (5) limited penetration to loci of infectious agents, (6) poor adherence in an aqueous form, low viscosity of previously known formulations, (7) long contact times required for antisepsis, (8) toxicity to the sinuses at high concentrations above 2.2% w/w, (9) poor tolerance by patients and inconvenient administration with currently available formulations. PVP-I has the desirable effect of inhibiting the formation of biofilms and to eliminate biofilms that have already formed. The efficacy and chemical stability of solutions of PVP-I is known to be highly dependent on concentration, pH, temperature, container system, solvent system and co-solvents.

DMSO has been shown to enhance the percutaneous penetration of many drugs. DMSO has also been shown to enhance the rate of penetration of water through the skin when the epidermis was treated for 30 minutes with 60%, 80% and 90% aqueous solutions of DMSO. Many theories concerning the mechanism of action of penetrants have appeared in the literature. One attributes the penetrant effects of DMSO, dimethylformamide, and dimethylacetamide to their hygroscopic properties which increase the water content of the stratum corneum, thereby greatly increasing its permeability. Reports of the efficacy of DMSO as a skin penetration enhancement agent require the use of high concentrations of DMSO typically above 50% and long contact times of at least 10-30 minutes. DMSO is known to be most effective with the use of only small molecules and, in this context, PVP-I is not considered a small molecule.

The present invention overcomes limitations in the prior art by providing an improved antiseptic composition, without steroids, with improved efficacy against sinusitis, rhinosinusitis, chronic sinus disease associated with cystic fibrosis even when used for very short contact times. The present invention overcomes limitations in the prior art by providing (1) an improved antiseptic efficacy for compositions with increased viscosity even when used for very short contact times and (2) an improved antiseptic efficacy for compositions with increased viscosity. Therefore, the compositions and methods disclosed, even when used for very short contact times, are substantially less irritating to human tissues than other antiseptics that contain higher concentrations of PVP-I.

The inventor has made the surprising discovery that the inclusion of a broad concentration of DMSO as a co-solvent, in a range of 2% to 30%, with water results in a dramatic improvement in the antimicrobial properties of iodine-based antiseptics even in the absence of alcohols. That is, the DMSO concentration may be, for example, 2%-3%, 3%-4%, 4%-5%, 5%-6%, 6%-7%, 7%-8%, 8%-9%, 9%-10%, 10%-12%, 12%-15%, 15%-20%, 20%-25%, 25%-30%, or 30%-35% or any combination thereof.

The inventor has also made the surprising discovery that the inclusion of cellulosic polymers, in a range of 0.1%-5%, 0.1%-4%, 0.1%-3%, 0.1%-2%, or 0.1%-1%, or any combination thereof, with water results in a dramatic improvement in the antimicrobial properties of iodine-based antiseptics even in the absence of alcohols and even in the absence of DMSO. It has further been discovered that these improved antiseptic compositions are able to eliminate bacterial infections, fungal infections, biofilm infections, virus infections, and the replication of viruses located within the sub-epithelial skin space, the sinuses and the nasal passages by treating only the surface of the skin, sinuses or nasal passages. Surprisingly, it has been discovered that these solutions are also active against the SARS-CoV-2 virus at specific concentrations and contact times.

It has been further discovered that even after treatments of the sinuses, nasal passages or surface skin for very brief contact times of less than 2 minutes, less than 1 minute, less than 30 seconds, less than 20 seconds, less than 15 seconds, less than 10 seconds and less than 5 seconds are effective for decontamination of the surface and even the sub-surface. It is further found that surprisingly short contact times as described above in this paragraph, can completely decontaminate the surfaces of all viruses, all bacteria, all microorganisms, and the sinusitis, rhinosinusitis, chronic sinus disease associated with cystic fibrosis virus.

One embodiment of the present invention employs a combination of penetration enhancer (e.g., DMSO), and antiseptic ingredient(s) (e.g., povidone-iodine), in order to deliver an effective therapeutic dose of a medicament. In any aspect of this disclosure, water is not considered a penetration enhancer. We note that the antiseptic ingredient may be the antiseptic class of iodophor agents. The iodophor agents (e.g., PVP-I), with dimethylsulfoxide employed as penetration enhancers and cellulosic polymers employed as viscosity enhancing agents was found to provide antimicrobial synergy and is one preferred embodiment.

The composition, in any method or embodiment, may comprise PVP-I in the following range and concentrations: about 0.01% to about 15%, about 0.05% to 12.5%, about 0.05% to 10.0%, about 0.1% to 10.0%, about 0.1% to 1.0%, about 0.15% to 1.5%, about 0.25% to 0.5%, about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00%, about 1.1%, about 1.2%, about 1.3%, about 1.4%, about 1.5%, or any range determinable from the preceding percentages including any range between any two values listed above. All percentages in this disclosure, for any compound or ingredients, unless otherwise specified, are expresses as weight percent (wt. %) based on total weight.

In one embodiment, the invention does not require DMSO to be present. Such a strategy is ideal because it allows polyantimicrobial agents to quickly and efficiently be delivered to the skin, sinuses, nasal passages or any other human surface in an effective, non-toxic concentration via a safe and convenient route. It is surprisingly shown that when these agents are combined with hydroxyethylcellulose, the antiviral efficacy against SARS-CoV-2 is enhanced in a synergistic fashion.

Another aspect of the invention is the development of compositions that include DMSO and members of the iodophor antiseptic class, including for example, PVP-I, with surprisingly improved stability when combined with cellulosic polymers. Such a composition is effective for PVP-I concentrations as low as 0.5% or as high at 10% on a w/w % basis. In additional examples, it was surprisingly found that even compositions with low concentrations of DMSO were able to penetrate the nasal epithelium and effectively arrest viral replication in the nasal passages, sinuses and other airway tissues in patients with CF-CRS. This is surprising because the action of PVP-I is taught to require very high concentrations of DMSO, such as above about 40% DMSO. We have discovered that these novel DMSO PVP-I systems with DMSO as low as 2-4% can disrupt viral replication intracellularly where conventional PVP-I aqueous solutions without PVP-I cannot reach the intracellular virus.

In certain embodiments, the composition may comprise less than about 30% DMSO, less than 25% DMSO, less than about 20% DMSO, less than about 15% DMSO, less than about 10% DMSO, less than about 5% DMSO, less than 4% DMSO, or about 4% DMSO. As another example, DMSO may be in the range of 1%-30%, 1%-25%, 1%-20%, 1%-15%, 1%-10%, 1%-5%, 1%-4%, about 4%, 3%-5%.

Surprisingly, even at these low concentrations of DMSO, in compositions with PVP-I and cellulosic polymers, these antiseptic solutions are able to penetrate through the human skin, mucosal, nasal, sinus and epithelial barriers to disrupt bacterial, viral, biofilm and other infections.

The methods and compositions of the invention are surprisingly useful for the treatment of sinusitis, rhinosinusitis, chronic sinus disease associated with cystic fibrosis infection of the nasal passages, upper airway, lower airway and other human tissues. The methods and compositions of the invention are also surprisingly useful or the treatment of surfaces after very short contact times of between 5 seconds to 10 seconds, 10 seconds to 15 seconds, 15 seconds to 20 seconds, 20 seconds to 30 seconds, 30 seconds to 45 seconds, and 45 seconds to 60 seconds.

A specific but non-limiting example of a formulation that leads to a useful pharmaceutical preparation consists of solid PVP-I dissolved or suspended in a 5% DMSO aqueous solution.

In another embodiment, DMSO can be added to aqueous solutions of PVP-I to produce solutions with DMSO concentrations in the range of 5%, 10%, 15%, 20%, 25%, 30%, 35%, 5% to 10%, 10% to 15%, 15% to 20%, 20% to 25%, 25% to 30%, or 30% to 35%. For example, DMSO can be present as a co-solvent with water in the range of 2%-40% DMSO. One embodiment of such a formulation could include a range of excipients such as sodium chloride, sodium dihydrogen phosphate monohydrate, cellulosic polymers like hydroxyethylcellulose, disodium hydrogen phosphate anhydrous and water, as well as others known to those skilled in the art.

In an additional embodiment, 10% PVP-I (w/v, aqueous) can be added to concentrations of DMSO aqueous solutions from 1-30% to yield a resulting solution of 1% PVP-I (w/w) with DMSO.

It is known that PVP-I aqueous solutions are difficult to stabilize at low concentrations over a long time. At concentrations less than about 0.7% PVP-I (w/w, aqueous), PVP-I aqueous solutions rapidly decay to yield complex mixtures of iodinated and iodine-free constituents. It is surprisingly found that in the aprotic DMSO solvent system employed in this invention, PVP-I solutions as low as 0.1% can be easily prepared and maintained as stable compositions, as determined by the USP method for determining the stability of PVP-I aqueous solutions, for long periods of time. It is of additional novelty that even in the hydrated DMSO solutions prepared from aqueous PVP-I, increased stability is observed for the PVP-I component.

It is particularly useful for the case of sinusitis, rhinosinusitis, chronic sinus disease associated with cystic fibrosis infections that stable, anhydrous or aqueous compositions that contain between 0.01%-2.5% PVP-I can be prepared in pure USP-grade DMSO solvents and in DMSO aqueous solutions of between 1% and 20%.

We note that in the following clinical examples (see Examples section), the formulations as described are able to alleviate signs and symptoms of CF-CRS and also in many cases are able to eradicate infections in CF-CRS including biofilms. This was, to us, a surprising and unexpected result.

The method of using the compositions described involves applying the compositions to the sinuses, nasal passages or other human tissue surfaces by rinsing with irrigation, topical administration, inhalation, nasal mist, or motorized nebulizer/atomizer/nasal lavage. The management of CF-CRS necessitates aggressive care of the sinonasal cavity to reduce biofilm burden and bacterial colonization of the sinuses. This requires regular outpatient debridement of the sinonasal cavity as well as application of topical PVP-I gel within the dependent sinuses (maxillary and sphenoid) post debridement to reduce biofilm formation. Furthermore, patients can continually use PVP-I with their daily regimen of nasal irrigation with isotonic/hypertonic solution using nasal nebulizer or lavage. Nasal spray of PVP-I will be frequently used by a patient when engaging healthcare providers to reduce the introduction of common resistant bacteria found within hospitals and clinics.

The invention described is surprisingly able to improve signs and symptoms of sinus disease. In one embodiment, acute signs and symptoms of disease are improved. In another embodiment, chronic signs and symptoms are improved in cases of sinus disease. In another embodiment, signs and symptoms of overall disease, disease-specific quality of life scores for patients, subjective score-based assessment of signs and symptoms and objective score-based signs and symptoms including also the improvement noted in the following categories:

1. Improvement of SNOT-22 score showing improving nasal symptoms and quality of life.
2. Improvement in the overall biofilm and culture of chronic sinonasal colonizing pathogens therefore the need for IV antibiotics.
3. Reduction of inflammatory edema, polyps and discharge graded by (Modified Lund-Kennedy endoscopic grading score) within the sinonasal cavity-causing obstruction of the sinus drainage pathway and stasis of thick tenacious mucus.
4. Improvement of overall sinus aeration as evaluated by CT scan and graded by (Lund-Mackay score).
5. The need for systemic steroid use to reduce inflammation associated with sinus infections given the potential severe adverse effects particularly in CF patients where pancreatic function is often compromised and diabetes is present.
6. Reduction of frequent upper respiratory viral infection.
7. The pulmonary function test score of patients will improve due to improvement of sinonasal chronic infection control.
8. Overall reduction of frequency of hospitalization due to reduction of lower airway exacerbation.

While the foregoing written description enables one of ordinary skill in the art to reproduce and use what is considered presently to be the best mode thereof, one of ordinary skill in the art will understand and appreciate the existence of variations, combinations, derivatives, analogs and equivalents of the specific embodiments, methods and examples provided above. The invention should, therefore, not be limited by the embodiments described herein, examples and methods by instead by all embodiments, examples and methods within the scope and spirit of the present invention.

Throughout this application, the term “about” is used to indicate that a value includes the inherent variation of error for the device, the method being employed to determine the value, or the variation that exists among the study subjects. The terms patient and subject are used interchangeably in this disclosure and have the same meaning. A preferred patient or subject is a mammal such as a human being.

The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

If unspecified, any composition in this disclosure may be in an aqueous solution with H₂O filling the remaining weight to 100%. If unspecified, any percentages in this disclosure may be in weight percent (wt. %).

The composition and methods of this disclosure are applicable for the treatment and disinfection of many microorganisms. Non-limiting examples of each type of microorganism are listed below.

The virus is preferably one that infects human. The human virus is preferably selected from an adenovirus, an astrovirus, a hepadnavirus, a herpesvirus, a papovavirus, a poxvirus, an arenavirus, a bunyavirus, a calcivirus, a coronavirus, a filovirus, a flavivirus, an orthomyxovirus, a paramyxovirus, a picornavirus, a reovirus, a retrovirus, a rhabdovirus, or a togavirus. In preferred embodiments, the adenovirus includes, but is not limited to, a human adenovirus. In preferred embodiments, the astrovirus includes, but is not limited to, a mamastrovirus. In preferred embodiments, the hepadnavirus includes, but is not limited to, the hepatitis B virus. In preferred embodiments, the herpesvirus includes, but is not limited to, a herpes simplex virus type I, a herpes simplex virus type 2, a human cytomegalovirus, an Epstein-Barr virus, a varicella zoster virus, a roseolovirus, and a Kaposi's sarcoma-associated herpesvirus. In preferred embodiments, the papovavirus includes, but is not limited to, human papilloma virus and a human polyoma virus. In preferred embodiments, the poxvirus includes, but is not limited to, a variola virus, a vaccinia virus, a cowpox virus, a monkeypox virus, a smallpox virus, a pseudocowpox virus, a papular stomatitis virus, a tanapox virus, a yaba monkey tumor virus, and a molluscum contagiosum virus. In preferred embodiments, the arenavirus includes, but is not limited to lymphocytic choriomeningitis virus, a lassa virus, a machupo virus, and a junin virus. In preferred embodiments, the bunyavirus includes, but is not limited to, a hanta virus, a nairovirus, an orthobunyavirus, and a phlebovirus. In preferred embodiments, the calcivirus includes, but is not limited to, a vesivirus, a norovirus, such as the Norwalk virus and a sapovirus. In preferred embodiments, the coronavirus includes, but is not limited to, a human coronavirus (e.g., SARS-CoV-2). In preferred embodiments, the filovirus includes, but is not limited to, an Ebola virus and a Marburg virus. In preferred embodiments, the flavivirus includes, but is not limited to, a yellow fever virus, a West Nile virus, a dengue fever virus, a hepatitis C virus, a tick borne encephalitis virus, a Japanese encephalitis virus, a Murray Valley encephalitis virus, a St. Louis encephalitis virus, a Russian spring-summer encephalitis virus, a Omsk hemorrhagic fever virus, a bovine viral diarrhea virus, a Kyasanus Forest disease virus, and a Powassan encephalitis virus. In preferred embodiments, the orthomyxovirus includes, but is not limited to, influenza virus type A, influenza virus type B, and influenza virus type C. In preferred embodiments, the paramyxovirus includes, but is not limited to, a parainfluenza virus, a rubula virus (mumps), a morbillivirus (measles), a pneumovirus, such as a human respiratory syncytial virus, and a subacute sclerosing panencephalitis virus. In preferred embodiments, the picornavirus includes, but is not limited to, a poliovirus, a rhinovirus, a coxsackievirus A, a coxsackievirus B, a hepatitis A virus, an echovirus, and an eneterovirus. In preferred embodiments, the reovirus includes, but is not limited to, a Colorado tick fever virus and a rotavirus. In preferred embodiments, the retrovirus includes, but is not limited to, a lentivirus, such as a human immunodeficiency virus, and a human T-lymphotrophic virus (HTLV). In preferred embodiments, the rhabdovirus includes, but is not limited to, a lyssavirus, such as the rabies virus, the vesicular stomatitis virus and the infectious hematopoietic necrosis virus. In preferred embodiments, the togavirus includes, but is not limited to, an alphavirus, such as a Ross river virus, an O'nyong' nyong virus, a Sindbis virus, a Venezuelan equine encephalitis virus, an Eastern equine encephalitis virus, and a Western equine encephalitis virus, and a rubella virus.

Non-limiting examples of bacteria that may be treated or disinfected include Escherichia sp., Staphylococcus sp., Thermus sp., Propionibacterium sp., Rhodococcus sp., Panninobacter sp., Caulobacter sp., Brevundimonas sp., Asticcacaulis sp., Sphingomonas sp., Rhizobium sp., Ensifer sp., Bradyrhizobium sp., Tepidimonas sp., Tepidicella sp., Aquabacterium sp., Pelomonas sp., Alcaligenis sp., Achromobacter sp., Ralstonia sp., Limnobacter sp., Massilia sp., Hydrogenophaga sp., Acidovorax sp., Curvibacter sp., Delftia sp., Rhodoferax sp., Alishewanella sp., Stenotrophomonas sp., Dokdonella sp., Methylosinus sp., Hyphomicrobium sp., Methylosulfomonas sp., Methylobacteria sp., Pseudomonas sp., Enterococcus sp., Myroides sp., Burkholderia sp., Alcaligenes sp. Specific examples include Escherichia coli, Staphylococcus aureus, Pseudomonas putida, Pseudomonas mendocina, Pseudomonas oleovorans, Pseudomonas fluorescens, Pseudomonas alcaligenes, Pseudomonas pseudoalcaligenes, Pseudomonas entomophila, Pseudomonas syringae, Methylobacterium extorquens, Methylobacterium radiotolerants, Methylobacterium dichloromethanicum, Methylobacterium organophilu, Hyphomicrobium zavarzini, Enterococcus faecalis, Myroides odoratus, Pseudomonas aeruginosa, Pseudomonas orizyhabitans, Burkholderia cepacia, Alcaligenes faecalis and Sphingomonas paucimobilis.

Non-limiting examples of fungus that can be treated or disinfected include Acremonium sp., Alternaria sp., Aspergillus sp., Cladosporium sp., Fusarium sp., Mucor sp., Penicillium sp., Rhizopus sp., Stachybotrys sp., Trichoderma sp., Dematiaceae sp., Phoma sp., Eurotium sp., Scopulariopsis sp., Aureobasidium sp., Monilia sp., Botrytis sp., Stemphylium sp., Chaetomium sp., Mycelia sp., Neurospora sp., Ulocladium sp., Paecilomyces sp., Wallemia sp., Curvularia sp.

Non-limiting examples of other microorganisms that can be treated or disinfected include Saccharomycotina, Taphrinomycotina, Schizosaccharomycetes, Basidiomycota, Agaricomycotina, Tremellomycetes, Pucciniomycotina, Microbotryomycetes, Candida sp. such as Candida albicans, Candida tropicalis, Candida stellatoidea, Candida glabrata, Candida krusei, Candida guilliermondii, Candida viswanathii, Candida lusitaniae and mixtures thereof, Yarrowia sp. such as Yarrowia lipolytica, Cryptococcus sp. such as Cryptococcus gattii and Cryptococcus neofarmans, Zygosaccharomyces sp., Rhodotorula sp. such as Rhodotorula mucilaginosa.

Other non-limiting examples of microorganisms suitable for the compositions and methods include those discussed in this disclosure.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

In various embodiments, the compositions encompassed herein comprise pharmaceutically acceptable excipients such as those listed in REMINGTON: THE SCIENCE AND PRACTICE OF PHARMACY 866-885 (Alfonso R. Gennaro ed. 19th ed. 1995; Ghosh, T. K.; et al. TRANSDERMAL AND TOPICAL DRUG DELIVERY SYSTEMS (1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

### INCORPORATION BY REFERENCE

All publications, patent applications, and patents mentioned herein are hereby incorporated by reference in their entirety as if each individual publication or patent was specifically and individually indicated to be incorporated by reference. In case of conflict, the present application, including any definitions herein, will control.

### EXAMPLES

Examples of Compositions Useful For The Treatment of Sinus Disease

### Example 1

1.0% PVP-I; 0.25% HEC; 2.0% DMSO; NaOH or other base to adjust pH to between 4-7; q.s. H₂O.

### Example 2

1.0% PVP-I; 0.50% HEC; 4.0% DMSO; NaOH or other base to adjust pH to between 4-7; q.s. H₂O.

### Example 3

1.0% PVP-I; 1.0% HEC; 6.0% DMSO; NaOH or other base to adjust pH to between 4-7; q.s. H₂O.

### Example 4

1.0% PVP-I; 1.25% HEC; 4.0% DMSO; NaOH or other base to adjust pH to between 4-7; q.s. H₂O.

### Example 5

1.0% PVP-I; 1.5% HEC; 10.0% DMSO; NaOH or other base to adjust pH to between 4-7; q.s. H₂O.

### Example 6

1.0% PVP-I; 1.75% HEC; 10.5% DMSO; NaOH or other base to adjust pH to between 4-7; q.s. H₂O.

### Example 7

1.0% PVP-I; 2.25% HEC; 11.0% DMSO; NaOH or other base to adjust pH to between 4-7; q.s. H₂O.

### Example 8

1.5% PVP-I; 0.50% HEC; 4.0% DMSO; NaOH or other base to adjust pH to between 4-7; q.s. H₂O.

### Example 9

1.5% PVP-I; 1.0% HEC; 5.0% DMSO; NaOH or other base to adjust pH to between 4-7; q.s. H₂O.

### Example 10

1.5% PVP-I; 1.25% HEC; 4.0% DMSO; NaOH or other base to adjust pH to between 4-7; q.s. H₂O.

### Example 11

1.5% PVP-I; 1.5% HEC; 10.0% DMSO; NaOH or other base to adjust pH to between 4-7; q.s. H₂O.

### Example 12

1.5% PVP-I; 1.75% HEC; 10.5% DMSO; NaOH or other base to adjust pH to between 4-7; q.s. H₂O.

### Example 13

2.0% PVP-I; 0.25% HEC; 2.0% DMSO; NaOH or other base to adjust pH to between 4-7; q.s. H₂O.

### Example 14

2.0% PVP-I; 0.50% HEC; 4.0% DMSO; NaOH or other base to adjust pH to between 4-7; q.s. H₂O.

### Example 15

2.0% PVP-I; 1.0% HEC; 6.0% DMSO; NaOH or other base to adjust pH to between 4-7; q.s. H₂O.

### Example 16

2.0% PVP-I; 1.25% HEC; 4.0% DMSO; NaOH or other base to adjust pH to between 4-7; q.s. H₂O.

### Example 17

2.0% PVP-I; 1.5% HEC; 10.0% DMSO; NaOH or other base to adjust pH to between 4-7; q.s. H₂O.

### Example 18

2.0% PVP-I; 1.75% HEC; 10.5% DMSO; NaOH or other base to adjust pH to between 4-7; q.s. H₂O.

### Example 19

2.0% PVP-I; 2.25% HEC; 11.0% DMSO; NaOH or other base to adjust pH to between 4-7; q.s. H₂O.

### Example 20

2.5% PVP-I; 0.50% HEC; 4.0% DMSO; NaOH or other base to adjust pH to between 4-7; q.s. H₂O.

### Example 21

2.5% PVP-I; 1.0% HEC; 5.0% DMSO; NaOH or other base to adjust pH to between 4-7; q.s. H₂O.

### Example 22

2.5% PVP-I; 1.25% HEC; 5.0% DMSO; NaOH or other base to adjust pH to between 4-7; q.s. H₂O.

### Example 23

2.5% PVP-I; 1.5% HEC; 10.0% DMSO; NaOH or other base to adjust pH to between 4-7; q.s. H₂O.

### Example 24

2.5% PVP-I; 1.75% HEC; 10.5% DMSO; NaOH or other base to adjust pH to between 4-7; q.s. H₂O.

### Example 25

1.0% PVP-I; 0.25% HEC; NaOH or other base to adjust pH to between 4-7; q.s. H₂O.

### Example 26

1.0% PVP-I; 0.50% HEC; NaOH or other base to adjust pH to between 4-7; q.s. H₂O.

### Example 27

1.0% PVP-I; 1.0% HEC; NaOH or other base to adjust pH to between 4-7; q.s. H₂O.

### Example 28

1.0% PVP-I; 1.25% HEC; NaOH or other base to adjust pH to between 4-7; q.s. H₂O.

### Example 29

1.0% PVP-I; 1.5% HEC; NaOH or other base to adjust pH to between 4-7; q.s. H₂O.

### Example 30

1.0% PVP-I; 1.75% HEC; NaOH or other base to adjust pH to between 4-7; q.s. H₂O.

### Example 31

1.0% PVP-I; 2.25% HEC; NaOH or other base to adjust pH to between 4-7; q.s. H₂O.

### Example 32

1.5% PVP-I; 0.50% HEC; NaOH or other base to adjust pH to between 4-7; q.s. H₂O.

### Example 33

1.5% PVP-I; 1.0% HEC; NaOH or other base to adjust pH to between 4-7; q.s. H₂O.

### Example 34

1.5% PVP-I; 1.25% HEC; NaOH or other base to adjust pH to between 4-7; q.s. H₂O.

### Example 35

1.5% PVP-I; 1.5% HEC; NaOH or other base to adjust pH to between 4-7; q.s. H₂O.

### Example 36

1.5% PVP-I; 1.75% HEC; NaOH or other base to adjust pH to between 4-7; q.s. H₂O.

### Example 37

2.0% PVP-I; 0.25% HEC; NaOH or other base to adjust pH to between 4-7; q.s. H₂O.

### Example 38

2.0% PVP-I; 0.50% HEC; NaOH or other base to adjust pH to between 4-7; q.s. H₂O.

### Example 39

2.0% PVP-I; 1.0% HEC; NaOH or other base to adjust pH to between 4-7; q.s. H₂O.

### Example 40

2.0% PVP-I; 1.25% HEC; 4.0% DMSO; NaOH or other base to adjust pH to between 4-7; q.s. H₂O.

### Example 41

2.0% PVP-I; 1.5% HEC; NaOH or other base to adjust pH to between 4-7; q.s. H₂O.

### Example 42

2.0% PVP-I; 1.75% HEC; NaOH or other base to adjust pH to between 4-7; q.s. H₂O.

### Example 43

2.0% PVP-I; 2.25% HEC; NaOH or other base to adjust pH to between 4-7; q.s. H₂O.

### Example 44

2.5% PVP-I; 0.50% HEC; NaOH or other base to adjust pH to between 4-7; q.s. H₂O.

### Example 45

2.5% PVP-I; 1.0% HEC; NaOH or other base to adjust pH to between 4-7; q.s. H₂O.

### Example 46

2.5% PVP-I; 1.25% HEC; NaOH or other base to adjust pH to between 4-7; q.s. H₂O.

### Example 47

2.5% PVP-I; 1.5% HEC; NaOH or other base to adjust pH to between 4-7; q.s. H₂O.

### Example 48

3.0% PVP-I; 0.25% HEC; NaOH or other base to adjust pH to between 4-7; q.s. H₂O.

### Example 49

3.0% PVP-I; 0.50% HEC; NaOH or other base to adjust pH to between 4-7; q.s. H₂O.

### Example 50

3.0% PVP-I; 1.0% HEC; NaOH or other base to adjust pH to between 4-7; q.s. H₂O.

### Example 51

3.0% PVP-I; 1.25% HEC; NaOH or other base to adjust pH to between 4-7; q.s. H₂O.

### Example 52

3.0% PVP-I; 1.5% HEC; NaOH or other base to adjust pH to between 4-7; q.s. H₂O.

### Example 53

3.0% PVP-I; 1.75% HEC; NaOH or other base to adjust pH to between 4-7; q.s. H₂O.

### Example 54

3.0% PVP-I; 2.25% HEC; NaOH or other base to adjust pH to between 4-7; q.s. H₂O.

### Example 55

3.5% PVP-I; 0.50% HEC; NaOH or other base to adjust pH to between 4-7; q.s. H₂O.

### Example 56

3.5% PVP-I; 1.0% HEC; NaOH or other base to adjust pH to between 4-7; q.s. H₂O.

### Example 57

3.5% PVP-I; 1.25% HEC; NaOH or other base to adjust pH to between 4-7; q.s. H₂O.

### Example 58

3.5% PVP-I; 1.5% HEC; NaOH or other base to adjust pH to between 4-7; q.s. H₂O.

### Example 59

3.5% PVP-I; 1.75% HEC; NaOH or other base to adjust pH to between 4-7; q.s. H₂O.

### Example 60

4.0% PVP-I; 0.25% HEC; NaOH or other base to adjust pH to between 4-7; q.s. H₂O.

### Example 61

4.0% PVP-I; 0.50% HEC; NaOH or other base to adjust pH to between 4-7; q.s. H₂O.

### Example 62

4.0% PVP-I; 1.0% HEC; NaOH or other base to adjust pH to between 4-7; q.s. H₂O.

### Example 63

4.0% PVP-I; 1.25% HEC; 4.0% DMSO; NaOH or other base to adjust pH to between 4-7; q.s. H₂O.

### Example 64

4.0% PVP-I; 1.5% HEC; NaOH or other base to adjust pH to between 4-7; q.s. H₂O.

### Example 65

4.0% PVP-I; 1.75% HEC; NaOH or other base to adjust pH to between 4-7; q.s. H₂O.

### Example 66

4.0% PVP-I; 2.25% HEC; NaOH or other base to adjust pH to between 4-7; q.s. H₂O.

### Example 67

4.5% PVP-I; 0.50% HEC; NaOH or other base to adjust pH to between 4-7; q.s. H₂O.

### Example 68

4.5% PVP-I; 1.0% HEC; NaOH or other base to adjust pH to between 4-7; q.s. H₂O.

### Example 69

4.5% PVP-I; 1.25% HEC; NaOH or other base to adjust pH to between 4-7; q.s. H₂O.

### Example 70

4.5% PVP-I; 1.5% HEC; NaOH or other base to adjust pH to between 4-7; q.s. H₂O.

### Example 71

4.5% PVP-I; 1.75% HEC; NaOH or other base to adjust pH to between 4-7; q.s. H₂O.

### Example 72

4.5% PVP-I; 1.75% HEC; NaOH or other base to adjust pH to between 4-7; q.s. H₂O.

### Example 73: Procedure A for Making a Composition for the Methods and Compositions of the Disclosure

All of the following examples are described at w/w % compositions with povidone-iodine (PVP-I) grade K30, hydroxyethylcellulose (HEC), DMSO (dimethylsulfoxide) and other components as listed. In another embodiment, all of the below examples can be prepared with similar w/w percent compositions of other suitable cellulosic polymers in place of HEC. In this disclosure, unless otherwise specified, it is understood that all solutions are

A generalized Procedure A, below, is described which can be used, along with other methods commonly known in the art, to produce the compositions listed in the Examples that contain DMSO.

Procedure A

1. In a one liter glass beaker, add dry povidone-iodine of desired quantity after calculating for hydration and correcting for 12 content based on specific lot certificate of analysis to quantity of DMSO and mix until dissolved.

2. While stirring at low speed, add H₂O up to 95% of desired amount.

3. Adjust pH as desired with NaOH or another base while stirring.

4. Incorporate HEC into the mixture using sieve addition with frequent mechanical mixing. Adjust speed of addition to ensure complete hydration without aggregation of HEC.

5. QS to the desired amount with H₂O.

6. Further adjust pH as desired with NaOH or another base while stirring.

7. As the hydration and gelation of HEC progress, increase the rate of mechanical stirring.

8. When solution is completely homogeneous remove from stirring for storage or packaging.

### Example 74: Procedure B for Making a Composition for the Methods and Compositions of the Disclosure

A generalized Procedure B, below, is described which can be used, along with other methods commonly known in the art, to produce the compositions listed in the Examples that contain DMSO.

Procedure B

1. In a one liter glass beaker, add dry povidone-iodine of desired quantity after calculating for hydration and correcting for 12 content based on specific lot certificate of analysis to quantity of H₂O and mix until dissolved.

2. While stirring at low speed, add H₂O up to 95% of desired amount.

3. Adjust pH as desired with NaOH or other base while stirring.

4. Incorporate HEC into the mixture using sieve addition with frequent mechanical mixing. Adjust speed of addition to ensure complete hydration without aggregation of HEC.

5. QS to the desired amount with H₂O.

6. Further adjust pH as desired with NaOH or another base while stirring.

7. As the hydration and gelation of HEC progress, increase the rate of mechanical stirring.

8. When solution is completely homogeneous remove from stirring for storage or packaging.

### Example 75: Examples of Patients Treated with Compositions Described in the Invention

Patient demographics are detailed in Table 1. None of the patients discontinued use due to intolerance. There were no reported adverse reactions to sinonasal irrigation with the PVP-I/HEC compositions that were used. Pre-treatment cultures were positive for 12/12 patients with multi-resistant species including MRSA, Enterococcus, Acenitobacter, Pseudomonas, Propionobacterium, S. viridans, Klebsiella and Serratia. Post-treatment cultures were all negative. No patients (0/12) had persistent positive cultures (Table 2).

TABLE 1
Patient Demographics

| | | | | PVPI/HEC/DMSO | Pre-Op | tPVP-I/ |
|---|---|---|---|---|---|---|
| Patient | Age | Gender | Diagnosis | Composition (w/w/w) | Treatment | HEC DMSO |
| 001 | 22 | M | CF | 1.25%/1.25%/0% | ESS/PO & IV Abx | 4 wks |
| 002 | 49 | F | CRS | 1.25%/1.25%/0% | rESS/PO Abx/ | 3 wks |
| 003 | 57 | M | CRS | 2.5%/1.0%/1.0% | ESS/PO Abx | 3 wks |
| 004 | 54 | F | CRS | 2.5%/1.0%/1.0% | ESS/PO Abx | 2 wks |
| 005 | 62 | F | CLL/L | 2.5%/1.25%/0% | Chemo/XRT | 3 wks |
| 006 | 46 | F | CRS | 2.5%/1.0%/3% | rESS/PO Abx | 2 wks |
| 007 | 58 | F | CRS | 1.25%/1.0%/0% | rESS/PO Abx | 2 wks |
| 008 | 27 | F | CF | 1.25%/1.25%/0% | ESS/PO & IV Abx | 3 wks |
| 009 | 51 | M | CRS | 1.25%/1.0%/0% | rESS/PO | 3 wks |
| 010 | 48 | M | CRS | 1.25%/1.0%/3.0% | ESS/PO &IV Abx | 4 wks |
| 011 | 39 | F | CRS | 1.25%/1.25%/1.0% | ESS/PO Abx | 2 wks |
| 012 | 55 | M | CRS | 1.25%/1.25%/1.0% | ESS/PO & IV Abx | 3 wks |

CRS: chronic rhinosinusitis.
CF: cystic fibrosis.
CLL: chronic lymphocytic leukemia.
L: lymphoma.
ESS: endoscopic sinus surgery.
rESS: revision endoscopic sinus surgery.
tPVP-I/HEC/DMSO duration of treatment with PVP-I/HEC/DMSO.
PO Abx: oral antibiotics.
IV Abx: intravenous antibiotics

TABLE 2
Clinical and Microbiological Data

| | PVPI/HEC/DMSO | | | | |
|---|---|---|---|---|---|
| Patient | Composition (w/w/w) | preCx | preSnOT | postCx | postSnOT |
| 001 | 1.25%/1.25%/0% | MRSA Ps | 24 | Neg | 17 |
| 002 | 1.25%/1.25%/0% | KO, Sa, SM | 61 | Neg | 15 |
| 003 | 2.5%/1.0%/1.0% | En, AB | 57 | Neg | 11 |
| 004 | 2.5%/1.0%/1.0% | MRSA, PA, SV | 71 | Neg | 34 |
| 005 | 2.5%/1.25%/0% | Sa | 53 | Neg | 36 |
| 006 | 2.5%/1.0%/3% | MRSA | 57 | Neg | 28 |
| 007 | 1.25%/1.0%/0% | Ps | 53 | Neg | 38 |
| 008 | 1.25%/1.25%/0% | Sa | 28 | Neg | 31 |
| 009 | 1.25%/1.0%/0% | Sa, SM, CK | 57 | Neg | 24 |
| 010 | 1.25%/1.0%/3.0% | Ps, MRSA | 61 | | 27 |
| 011 | 1.25%/1.25%/1.0% | Sa, SM, CK | 43 | Neg | 32 |
| 012 | 1.25%/1.25%/1.0% | Sa, SM, CK | 59 | Neg | 38 |

MRSA: methicillin resistant staph aureaus.
Ps: Pseudomonas auroginosa.
KO: Klebsiella oxytoca.
Sa: Staph aureaus.
SM: Serratia marcescens.
En: Enterococus Cloacae.
AB: acenitobacter Baumanni.
PA: Propionibacterium Acnes.
SV: Strep viridans.
CK: Citrobacter Koseri.
Neg: negative

We claim:

1. A composition for treating, controlling, reducing, inhibiting or preventing a chronic established biofilm in a sinus of a human when applied topically or through irrigation, the composition comprising 0.5 wt %-2.5 wt % povidone-iodine; 0.15 wt %-1.25 wt % hydroxyethylcellulose; and water.

2. The composition of claim 1 wherein the composition does not contain DMSO.

3. The composition of claim 1 wherein the composition consists of 0.5 wt %-2.5 wt % povidone-iodine; 0.15 wt %-1.25 wt % hydroxyethylcellulose; and water.

4. The composition of claim 1 which comprises a suitable amount of pharmaceutically acceptable halide-salt to make the solution iso-osmotic with nasal mucosa.

5. A method for treating, controlling, reducing, inhibiting or preventing a bacterial infection in a human, the method comprises the step of contacting a site of the bacterial infection with a composition of claim 1.

6. The method of claim 5, wherein the human has cystic fibrosis and chronic sinusitis associated with biofilm formation.

7. The method of claim 5 wherein the site is a sinus surface and wherein contacting comprises irrigating the sinus of the human with a composition of claim 1

8. The method of claim 7 wherein the bacterial infection is a chronic established biofilm in a sinus of a human.

9. A method for treating, controlling, reducing, inhibiting or preventing a virus infection in a human, the method comprises the step of contacting a site of the virus infection or potential virus infection with a composition of claim 1.

10. The method of claim 9 wherein the virus is SARS-CoV-2.

11. A composition for treating, controlling, reducing, or inhibiting a chronic established biofilm in a sinus of a human when applied topically or through irrigation, the composition comprises 0.5 wt %-2.5 wt % povidone-iodine; 0.15 wt %-1.25 wt % hydroxyethylcellulose; 0.1 wt % to 2.5 wt % DMSO, and water.

12. The composition of claim 11 wherein the composition consists of 0.5 wt %-2.5 wt % povidone-iodine; 0.15 wt %-1.25 wt % hydroxyethylcellulose; 0.1 wt % to 2.5 wt % DMSO, and water.

13. The composition of claim 11 which comprises a suitable amount of pharmaceutically acceptable halide-salt to make the solution iso-osmotic with nasal mucosa.

14. A method for treating, controlling, reducing, inhibiting or preventing a bacterial infection in a human, the method comprises the step of contacting a site of the bacterial infection with a composition of claim 11.

15. The method of claim 14 wherein the human has cystic fibrosis and chronic sinusitis associated with biofilm formation.

16. The method of claim 14 wherein the site is a sinus surface and wherein contacting comprises irrigating the sinus of the human with a composition of claim 11.

17. The method of claim 16 wherein the bacterial infection is a chronic established biofilm in a sinus of a human.

18. A method for treating, controlling, reducing, inhibiting or preventing a virus infection in a human, the method comprises the step of contacting a site of the virus infection or potential virus infection with a composition of claim 11.

19. The method of claim 18 wherein the virus is SARS-CoV-2.
